(19) United States
(12) Patent Application Publication
(10) Pub. No.: US 2017/0002349 A1
(43) Pub. Date: Jan. 5, 2017

(54) SMALL RNA, PREPARATION METHOD THEREFOR AND APPLICATION THEREOF IN PHARMACEUTICALS FOR SPECIFICALLY UP-REGULATING GENE TRANSCRIPTIONAL ACTIVITY

(71) Applicant: SUN YAT-SEN UNIVERSITY, Guangzhou, Guangdong (CN)

(72) Inventors: Hui Zhang, Guangzhou (CN); Yijun Zhang, Guangzhou (CN)

(73) Assignee: Sun Yat-Sen University, Guangzhou (CN)

(21) Appl. No.: 15/028,328

(22) PCT Filed: Oct. 9, 2013
(86) PCT No.: PCT/CN2013/084908

§ 371 Date: Apr. 8, 2016

(51) Int. Cl. C12N 15/113

(52) U.S. Cl. CPC C12N 15/113; C12N 15/1136; C12N 2310/141; C12N 2310/14

(57) ABSTRACT

Disclosed in the present invention are a method for specifically up-regulating gene expression through targeting core promoter using small RNAs (including microRNAs and small interfering RNAs), and a series of regulation target genes. An objective of the present invention is to provide a method for specifically up-regulating gene expression using micro-RNAs, and the present invention possesses a high application value and a broad application prospect in the fields of biotechnology and biomedicine.

### TECHNICAL FIELD

The present invention relates to a field of small RNA, more particularly, to small RNA, preparation method therefor, and application thereof in specifically up-regulating gene transcriptional activity.

### BACKGROUND

Precise regulation of gene expression has important significance for the development of organisms, and dysregulation of genes may lead to many serious diseases such as cancers, immune diseases, neurological diseases or metabolic diseases (for example, diabetes) etc. Therefore, the techniques for precisely regulating gene expression are of great values of application in researches and clinical treatments. Currently, the method for regulating endogenous gene expression is mainly a technique called “RNA interference”. The technique of RNA interference uses a double-stranded RNA molecule that is homologous to a target gene, and uses the RNA silencing pathway to inhibit the target gene expression. Main members of the RNA silencing pathway are DICER protein and RNA-induced silencing complex (RISC), the main member of which is AGO protein. The double-stranded RNA molecule is firstly sliced into small segments of approximately 22 bp by DICER within cell, the antisense RNA segments of which are subsequently recruited into RISC to bind to the target mRNA and lead to its degradation. At present, the widely used RNA interfering means are siRNA and shRNA. SiRNA is a synthesized double-stranded RNA molecule having a general length of 19 nt and terminals modified with overhang, the antisense strand of which is complementary to target gene mRNA. ShRNA is generally a sequence homologous with a target gene inserted into an expression vector, which is processed into short siRNAs after expressed in cells. When shRNA is expressed by means of a lentiviral vector, gene suppression effects being stable for a long time can be achieved. However, only the effects of silencing or reducing endogenous gene expression can be achieved by the technique of RNA interference, and there is still no method for specifically and effectively up-regulating endogenous gene expression. Upregulating the expression of certain genes is of great values in life science researches and clinical treatment applications. For example, the enhancement of an amount of insulin expression in diabetic patients by upregulating its transcription may relieve their symptoms and improve their life quality.

MicroRNA (MiRNA) is a class of small-molecule regulatory RNA, that widely presents in animals, plants, fungi and even viruses. MiRNA is involved in a number of important regulatory pathways, whose dysregulation will induce many serious diseases. The regulatory pathway of miRNA is very similar to that of siRNA, both of which bind to the target mRNA in RISC for restraining the gene expression, except that the role of miRNA is mainly to restrain translation of mRNA, while siRNA directly leads to degradation of mRNA. Currently, it is widely considered that a regulating mechanism of miRNA is binding to the 3′ untranslated region (3′ UTR) of the target mRNA in RISC of cytoplasm to restrain translation of mRNA. In recent years, it is found that miRNAs are not only distributed in cytoplasm, some miRNAs are also highly enriched in the nucleus. However, the functions of these miRNAs are still unclear.

### SUMMARY OF THE INVENTION

One of the objects of the present invention is to provide a small RNA that can improve the transcriptional activity of interested gene.

Firstly provided as required is a preparation method for small RNAs for up-regulating endogenous gene expression, including the following steps:

S1. identification of target site of small RNA which is in the range of 20 bases extending upstream and downstream respectively from a TATA box sequence serving as a center, when the TATA box sequence is contained in a gene promoter, and which is sequence of 1-50 bases upstream from a starting site for the gene transcription, when the TATA box sequence isn't contained in the gene promoter.

S2. design and synthesis of small RNA

Antisense strand of the small RNA is complementary in sequence to the target site, to obtain a core sequence; and

S3. modifying the resultant core sequence of S2, so that the small RNA contains the following structures:

double-stranded siRNA having a length of 19 bases and a dTdT pendant at 3′ terminal, double-stranded siRNA having a length less than or more than 19 bases, microRNA synthesized in vitro, single-stranded or double-stranded antisense RNA synthesized in vitro, or chemically modified siRNA, microRNA and antisense RNA synthesized in vitro.

Further provided are applications of the small RNA obtained according to the above-described preparation method in regulating the transcriptional activity of gene and medicines thereof.

The regulating transcriptional activity of gene means interacting with the TATA box sequence or other core promoters to thereby enhance the expression of gene.

Further provided is a small RNA prepared according to the above-described method.

The small RNA is hsa-let-7i, hsa-miR-138, hsa-miR-92a, hsa-let-7c, or hsa-miR-181d,

a gene sequence of said hsa-let-7i is shown as SEQ NO: 1,

a gene sequence of said hsa-miR-138 is shown as SEQ NO: 2,

a gene sequence of said hsa-miR-92a is shown as SEQ NO: 3,

a gene sequence of said hsa-let-7c is shown as SEQ NO: 4, and

a gene sequence of said hsa-miR-181d is shown as SEQ NO: 5.

Finally provided are applications of the above-described small RNAs in regulating transcriptional activities of genes and medicines thereof.

The regulating transcriptional activities of genes means enhancing the transcriptional activities of genes of interleukin 2, insulin, calcitonin, histone, and c-myc.

S5. Provided is a small interfering RNA prepared according to the above-described method, which is IL-2TATAcen, INS-TATAcen, LHB-TATAcen, POMC-TATAcen, NPPA-TATAcen, IL6-TATAcen, HIV-TATAcen, H4A1-TATAup, APOE-TATAcen, CIRBP-TATAup, BCL2L12-TATAcen, RHO-TATAcen, CALCA-TATAup, GAPD-TATAdn, or HBB-TATAcen,

a gene sequence of said IL-2TATAcen is shown as SEQ NO: 6,

a gene sequence of said INS-TATAcen is shown as SEQ NO: 7,

a gene sequence of said LHB-TATAcen is shown as SEQ NO: 8,

a gene sequence of said POMC-TATAcen is shown as SEQ NO: 9,

a gene sequence of said NPPA-TATAcen is shown as SEQ NO: 10,

a gene sequence of said IL6-TATAcen is shown as SEQ NO: 11,

a gene sequence of said HIV-TATAcen is shown as SEQ NO: 12,

a gene sequence of said H4A1-TATAup is shown as SEQ NO: 13,

a gene sequence of said APOE-TATAcen is shown as SEQ NO: 14,

a gene sequence of said CIRBP-TATAup is shown as SEQ NO: 15,

a gene sequence of said BCL2L12-TATAcen is shown as SEQ NO: 16,

a gene sequence of said RHO-TATAcen is shown as SEQ NO: 17,

a gene sequence of said CALCA-TATAup is shown as SEQ NO: 18,

a gene sequence of said GAPD-TATAdn is shown as SEQ NO: 19, and

a gene sequence of said HBB-TATAcen is shown as SEQ NO: 20.

More further provided is an application of the above-described small interfering RNA in regulating transcriptional activities of genes.

The regulating transcriptional activities of genes means enhancing the transcriptional activities of genes of interleukin 2, insulin, LHB, POMC, NPPA, IL6, HIV-1 virus, H4A1, APOE, CIRBP, BCL2L12, RHO, CALCA, GAPD, and HBB.

Advantages of the present invention are as follows:

We find in studies that a large number of miRNAs in cells associated with polymerase II core transcription factors, and computational predictions indicate that many of these miRNAs could bind to the TATA box sequence of a gene promoter. Wherein, the miRNA hsa-let-7i can bind to the TATA box sequence of interleukin-2 gene promoter and enhance expression of the gene. Other miRNAs including hsa-let-7i, hsa-miR-138, hsa-miR-92a, hsa-let-7c and hsa-miR-181d specifically up-regulate the transcriptional activity of gene of interleukin-2, insulin, calcitonin, histone (H4A1) and c-myc respectively, by combining with TATA box sequence on the promoter. In addition, we find that the transcriptional activity of nearly 80% tested genes can be increased by using artificially-synthesized siRNAs targeting the gene TATA box.

1. The present invention provides evidences showing that the microRNA hsa-let-7i in human cells sequence-specifically targets the TATA box sequence of interleukin-2 (IL2) gene promoter, and enhances expression levels of IL-2 mRNA and proteins. Animal experiments on mice show that the miRNA mmu-let-7i of mice can enhance the expression levels of IL-2 in blood of mice.

2. The present invention provides evidences showing that enhancement of the expression levels of IL-2 by hsa-let-7i is sequence-specific, regulatory effect of which is in positive correlation to a complementarity between the sequences of microRNA and TATA box region of the gene promoter.

3. The present invention provides evidences showing that the miRNA is capable of combining directly with the TATA box of the gene promoter in the sequence, and leads to increased transcription initiation rate of gene.

4. The present invention provides evidences showing that intracellular miRNAs including hsa-miR-138, hsa-miR-92a, hsa-let-7c and hsa-miR-181d sequence-specifically up-regulate the transcription activities of genes of insulin, calcitonin, histone (H4A1) and c-myc by targeting the TATA box respectively.

5. The present invention provides evidences showing that three siRNAs designed in the range of 20 bp extending upstream and downstream respectively from the TATA box serving as a center can up-regulate the transcriptional activity of nearly 80% of the tested genes.

### BRIEF DESCRIPTION OF THE DRAWINGS

FIG. 1 shows combinations of a number of miRNAs with transcriptional complex of RNA polymerases in cells. (A) Combinations of a number of miRNAs with RNA polymerase II in Peripheral Blood Mononuclear Cells (PBMCs). (B) Identification results of microRNA chip. (C) Results of microRNA chip verified by real time fluorescent quantitative PCR. (D) Combination of microRNA with TATA box binding protein TBP verified by realtime fluorescent quantitative PCR.

FIG. 2 shows that CD4+ T cells of human and animal experiment on mice indicates hsa-let-7i being capable of enhancing the expression of interleukin-2 (IL-2). (A) Combining situations of hsa-let-7i with the IL-2 core promoter predicted by a computer. (B) The hsa-let-7i mimic can enhance the transcriptional activity of the IL-2 promoter. (C) The hsa-let-7 imimic can enhance the expression of the endogenous IL-2 mRNA in Jurkat cells. (D) The hsa-let-7i mimic can enhance the expression of the endogenous IL-2 mRNA in human primary CD4+ T cells. (E) The hsa-let-7i mimic can enhance the secretion of the endogenous IL-2 in human primary CD4+ T cells. (F) The hsa-let-7i mimic can enhance the expression of the endogenous IL-2 mRNA in mouse primary CD4+ T cells. (G) The hsa-let-7i mimic can enhance the expression levels of IL-2 in blood in mice body.

FIG. 3 shows that hsa-let-7i regulates the transcriptional activity of IL-2 by sequence-specifically targeting TATA box sequence. (A) Mutating the combining sites on IL-2 promoter influences the regulating effect of hsa-let-7i. (B) Mutating hsa-let-7i influences the regulating effect thereof on IL-2. (C) Respectively mutating the combining sites on hsa-let-7i and IL-2 promoter and rematching those two may restore the regulating effects of hsa-let-7i. (D) Effect of enhancing the transcriptional activity of IL-2 by other members of the let-7 family is positively correlated to the similarity thereof with hsa-let-7i sequence.

FIG. 4 shows that miRNA may combine with the promoter DNA and enhance the transcriptional activity of gene. (A) IL-2 core promoter may combine with hsa-let-7i, and this combination is affected when IL-2 core promoter sequence is mutated. (B) The combination of IL-2 core promoter with hsa-let-7i is affected by RNase H. (C) hsa-let-7i enhances the transcription initiation rate of IL-2 promoter. (D) hsa-let-7i enhances the transcriptional elongation process of IL-2 promoter.

FIG. 5 shows that miRNAs, hsa-miR-138, hsa-miR-92a, hsa-let-7c, and hsa-miR-181d enhance the transcriptional activities of the promoters of genes of insulin, calcitonin, histone (H4A1), and c-myc, by sequence-specifically combining TATA box region. (A) Computer predicts the combination situations of miRNAs with gene promoters, wherein hsa-miR-138, hsa-miR-92a, hsa-let-7c, and hsa-miR-181d combine with TATA box sequences of genes of insulin, calcitonin, histone (H4A1), and c-myc respectively. (B) miRNAs, hsa-miR-138, hsa-miR-92a, hsa-let-7c, and hsa-miR-181d up-regulate the transcriptional activities of the promoters of genes of insulin, calcitonin, histone (H4A1), and c-myc respectively.

FIG. 6 shows that artificially-synthesized small interfering RNAs targeting TATA box enhance effectively the transcriptional activities of the promoters. (A) The upper part shows schematic diagram of designing sites of siRNAs targeting TATA box sequence of the genes; and the lower part performs the tests by selecting randomly 19 promoters of genes containing TATA box sequences, the transcriptional activities of 15 (78.9%) genes of which can be enhanced in dual-luciferase reporter system by the artificially-synthesized siRNAs, including IL-2TATAcen, INS-TATAcen, LHB-TATAcen, POMC-TATAcen, NPPA-TATAcen, IL6-TATAcen, HIV-TATAcen, H4A1-TATAup, APOE-TATAcen, CIRBP-TATAup, BCL2L12-TATAcen, RHO-TATAcen, CALCA-TATAup, GAPD-TATAdn, or HBB-TATAcen. The artificially-synthesized siRNAs targeting IL-2 TATA box enhance the expression of the endogenous IL-2 mRNA (B) and protein (C).

FIG. 7 shows that small interfering RNAs targeting the core promoters of non-TATA box genes effectively enhance the transcriptional activities of the promoters and mRNA expression. (A) Effects of small interfering RNAs targeting the core promoters of nerve growth factor (NGF) and apolipoprotein B receptor (APOBR) of non-TATA box genes, on the transcriptional activities of genes. (B) Effects of siRNAs targeting the core promoters of non-TATA box genes Akt1, CDC25A, ERK2, Bmi-1 and JNK, on the expression of gene mRNAs.

### DETAILED DESCRIPTION

The present invention is further described in details in combination with drawings and the specific Examples below. Unless otherwise specified, reagents, equipments, and methods used in the present invention are all conventional and commercially available reagents, equipments, and conventionally used methods in this technical field.

### Example 1

### MiRNA hsa-let-7i Up-Regulate IL-2 Expression by Targeting TATA Box

1.1 Preparation Method

Plasmids, miRNA Mimics, and Interfering RNAs

A luciferase reporter vector promoted by a human IL-2 promoter is constructed by replacing the CMV promoter on pMIR-Reporter vector (Promega) with −400 to +1 bp section relative to the transcription starting site (TSS) of the human IL-2 promoter using restriction endonuclease. A PLKO-let-7i vector is constructed by inserting the precursor sequence of hsa-let-7i into PLKO.1 vector (Sigma), and replacing the puromycin gene on the vector with GFP. Wild-type hsa-let-7i, mmu-let-7i, mutant-type hsa-let-7i, agomir-hsa-let-7i and the corresponding negative controls are purchased from Genepharma, Shanghai, China or Ribobio, Guangzhou, China.

Antibodies and Reagents

Anti-HA (MMS-101P) and anti-Pol II (8WG16) monoclonal antibodies are purchased from Covance Inc., anti-TBP monoclonal antibody (ChIP Ab+) is purchased from Upstate (Millipore), anti-actin antibody (D6A8) is purchased from Cell Signaling Technology (CST, Danvers, Mass.), anti-human CD3 and anti-human CD28 antibodies are purchased from BD (Palo Alto, Calif.), and anti-mouse CD3 and anti-mouse CD4 are purchased from eBioscience (San Diego, Calif.). Phorbol myristate acetate (PMA) and ionomycin are purchased from Sigma. EZ-Magna ChIP A/G kit (10086) used for RNA-ChIP is purchased from Millipore.

Cell Culture

Jurkat and HEK293T cells are purchased from ATCC (American Type Culture Collection) and cultured according to the operation specification thereof. Human peripheral blood lymphocyte (PBMCs) are isolated from the whole blood of healthy human with Ficoll-Hypaque Solution, primary CD4+ T cells are then isolated therefrom with CD4+ T Cell Isolation Kit II (BD), and the PBMCs and the CD4+ T cells are both cultivated with RPMI 1640 containing 10% fetal bovine serum (FBS), 50 units/mL penicillin and 50 μg/mL streptomycin.

Transfection and Infection

The wild type hsa-let-7i and negative control of microRNA mimics are transfected into CD4+ T cells of human or mouse with Lipofectamine RNAiMAX (Invitrogen) at a final concentration of 50 nM. After 48-72 hours, stimulation is performed with anti-CD3 (1 μg/mL) and anti-CD28 (5 μg/mL) for 12-24 hours. Agomir-hsa-let-7i and the corresponding negative control are directly added to CD4+ T cells culture medium at a final concentration of 50 nM, and then stimulation is performed in the same method as described above. HEK293T cells are transfected with Lipofectamine 2000 (Invitrogen). Three plasmids of pCMV-ΔR8.2, VSV-G, and PLKO.1 are co-transfected into HEK293T cells (60% density) with Lipofectamine 2000. After 48 hours, supernatant containing lentivirus is collected, and 4 μg/mL polybrene is added to infect Jurkat cells. The infected GFP+ cells are sorted with a flow cell sorter (BD Bioscience, Palo Alto, Calif.), and they are stimulated with 5 ng/mL PMA and 1 μM ionomycin.

MicroRNA Chip and Data Analysis

Expression of miRNAs is examined with Serum/Plasma Focus miRNA PCR Panel. The data analysis is conducted as specified.

Real Time Quantitative RT-PCR Analysis

Total RNAs of Jurkat and CD4+ T cells are extracted with TRIzol reagent (Invitrogen), cDNAs are synthesized by performing reverse transcription with PrimeScript RT reagent Kit (Takara) according to the instruction book, and quantitative PCR was then performed with SYBR Premix ExTaq II Kit (Takara) according to the instruction book, with a housekeeping gene GAPDH or β-actin as an internal control.

Computational Predictions of miRNA and Target Site Thereof

The core promoter sequences (−49 to +1) of genes are downloaded from the Eukaryotic Promoter Database (EPD, http://epd.vital-it.ch/), wherein one containing “ATAA”, a conserved gene sequence is the core promoter containing a TATA box. The mature sequences of miRNAs are downloaded from miRBase (http://www:mirbase.org/). The target sites of miRNAs on core promoter sequences of genes are predicted with RNA-hybrid web server (http://bibiserv.techfak.uni-bielefeld.de/rnahybrid), and the target sites having a MFE value less than −25 kcal/mol are selected.

Dual-Luciferase Reporter Assay

Approximately 20,000 HEK293T cells are paved in each well of a 48-well plate one day before transfection. 5-10 ng of firefly luciferase reporter vector promoted by IL-2 promoter and 2 ng renilla luciferase vector are transfected into each well with Lipofectamine 2000, and miRNA precursor vector or control vector, or miRNA mimic or small interfering RNA or the corresponding negative control (a final concentration of 50 nM) are cotransfected. After 24-48 hours, the activities of dual-luciferase are measured with the Dual-Glo luciferase assay system (Promega).

Co-Immunoprecipitation of RNA-Chromatin

The human PBMCs are stimulated with anti-CD3 (1 μg/ml) and anti-CD28 (5 m/ml) for 48 h, washed once with pre-cooled PBS, cross-linked with 1% formaldehyde, subsequently stopped by adding 0.125M glycine, and ultrasonically lysed. The supernatants are taken, into which anti-RNA Pol II (5 μg), anti-TBP (5 μg), or seronegative mouse IgG (5 μg) and 50 μL protein A/G magnetic beads are added. The mixture is incubated overnight at 4° C. The incubated mixture is washed with 0.5 mL the following precooled buffers: low-salt washing liquid, high-salt washing liquid, LiCl washing liquid, and TE washing liquid (Millipore). RNAs are then extracted by phenol/chloroform/isoamyl alcohol. DNAs in RNAs are removed with TURBO DNA free kit (Ambion). Then, 1 μL of RNA, 1 μL of 32P-γ-ATP (3000 Ci/mmol, 10 mCi/mL, Perkin Elmer), 2 μL 10× kinase reaction buffer, 1 μl T4 polynucleotide kinase (Takara), and 15 μL dH₂O are mixed and incubated at 37° C. for 1 hours. The labeled RNAs are subject to denatured PAGE electrophoresis. Finally, exposure is performed using X-ray film for 3 hours.

Initial Rates Assay for Transcription of Gene

IL-2 promoter promoted firefly luciferase reporter vector and let-7i mimics or negative control (a final concentration of 50 nM) are transfected into HEK293T cells. After 20 h, cells are collected, into which 5 μL of 10 mCi/mL [α-32P] UTP is added. After treatment with DNase I and protease K, RNAs are extracted with phenol/chloroform/isoamyl alcohol and hybridized to fix on a nylon membrane overnight at 37° C. Approximately 200-bp probe of GAPDH or RFP act as sample loading and negative control, respectively. Components of hybridization liquid are: 50% formamide, 6×SSC, 10×Denhardt's solution, and 0.2% SDS. After washing the membrane with 2×SSC and 0.2% SDS for three times, exposure is performed with Phosphor Imager screen (GE Healthcare).

Combining Experiments In Vitro of microRNAs and the Core Promoters

20 μL reaction system is prepared at room temperature to assemble pre-initiation-complex (PIC) of RNA polymerase II by mixing the following components: 20 mM HEPES, 5 mM MgCl₂, 8% glycerol, 100 mM KCl, 4 μg acetylated BSA, 2 μg Poly(dI-dC)•Poly(dI-dC) (Sigma), 20 μg HeLa cell nuclear extracts, 0.4 pmol radioisotope-labeled double-stranded oligonucleotides, with an unlabeled double-stranded oligonucleotides of IL-2 core promoter as a competition. 2-5 μg specific antibody is added, and after reacting on ice for 1 hours, electrophoresis is performed with 4.5% undenatured PAGE at a condition of 150 V for 3.5 hours. When performing combination of leti-7i with IL-2 core promoter in vitro, radioisotope-labeled double-stranded oligonucleotides change into biotin-labeled IL-2 core promoter and non-specific double-stranded oligonucleotides. After incubating the assembled PIC for 15 minutes, the complex is added into 100 μg MyOne streptavidin C1 immunomagnetic beads (Invitrogen). Upon washing for 3 times, resuspension is performed with 20 μL buffer [1M NaCl, 5 mM Tris-HCl (pH 7.5), and 0.5 mM EDTA]. The incubation is performed at room temperature with slightly rotating for 15 minutes to fix PIC. The magnetic beads are washed at room temperature for 3 times with 0.5 mL 0.1×SSC/0.1% SDS, each for 10 minutes. Treatment with RNase H is performed at 37° C. for 10 minutes. microRNAs obtained by hybridization are dissolved using 40 μL eluent. RNAs are extracted with Trizol and subjected to realtime fluorescent quantitative reverse transcription PCR determination.

Experiments of Animals

The female BALB/cA mice (8-10 weeks) are treated with 25 nmol agomir-mmu-let-7i or negative control with injection of caudal vein. Determination of IL-2 levels in serum is performed 48 hours after the injection.

1.2 Structure

The structure of the resultant miRNA is as follows:

hsa-let-7i
5′ ugagguaguaguuugugcuguu 3′
mmu-let-7i
5′ ugagguaguaguuugugcuguu 3′

1.3 Confirmation of Effects and Application

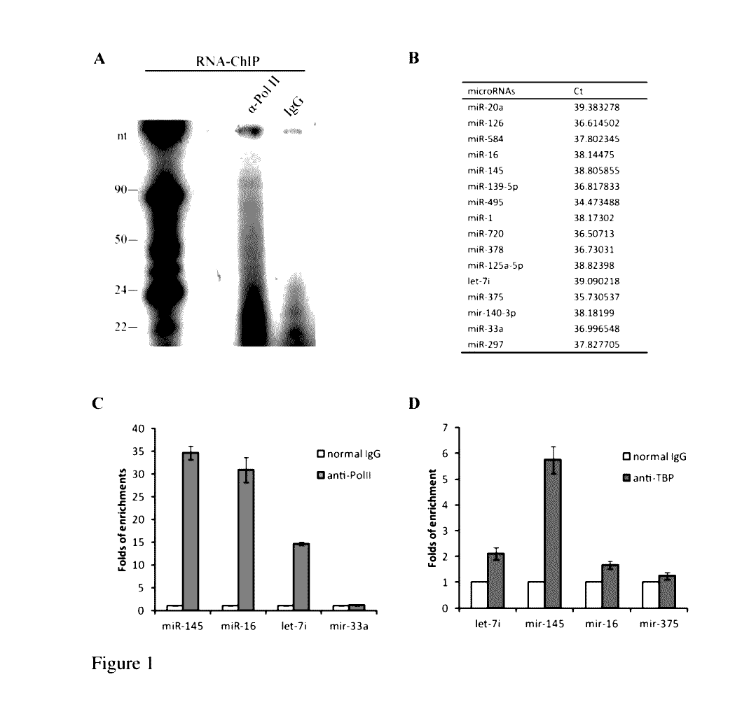

We found a large number of intracellular microRNAs combine with RNA polymerase II of polymerase II core transcription factors (FIG. 1A). MicroRNA chip analysis identifies many miRNA genes (FIG. 1B), including hsa-let-7i therein. In addition, we use real time fluorescent quantitative PCR to verify some of miRNAs combining with the RNA polymerase II or TATA box combining protein (TBP), including hsa-let-7i (FIGS. 1C and D) therein.

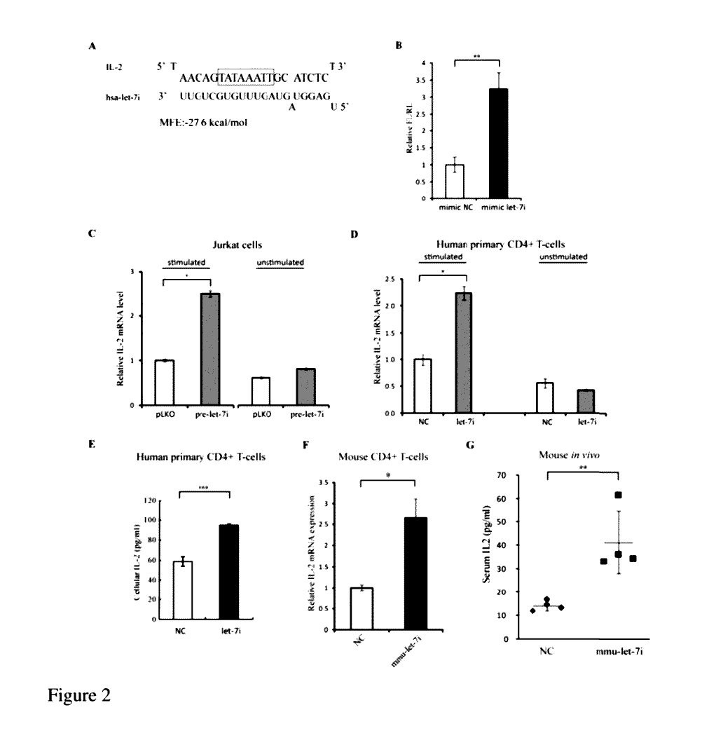

MiRNA hsa-let-7i sequence-specifically targets TATA box sequence of interleukin-2 (IL2) gene promoter and enhances mRNA and protein expression levels of IL-2. Animal experiments on mice show that miRNA mmu-let-7i of mouse can enhance expression levels of IL-2 in mouse blood. Computational predictions indicate that many of these miRNA therein could bind to the TATA box sequence of the gene promoter, wherein the miRNA hsa-let-7i is capable of binding to TATA box sequences of the IL-2 gene promoter (FIG. 2A). Results of dual-luciferase reporter show that hsa-let-7i enhances the transcriptional activity of IL-2 gene promoter (FIG. 2B). In lymphocytic Jurkat cells, hsa-let-7i enhances the expression of IL-2 mRNA (FIG. 2C). In primary CD4+ T cells, hsa-let-7i enhances the expression of IL-2 mRNA and protein (FIGS. 2D and E). In addition, in mouse primary CD4+ T cells, mm-let-7i up-regulates the expression of IL-2 mRNA (FIG. 2F). Animal experiments on mice show that mm-let-7i enhances the expression levels of IL-2 in mouse blood (FIG. 2G).

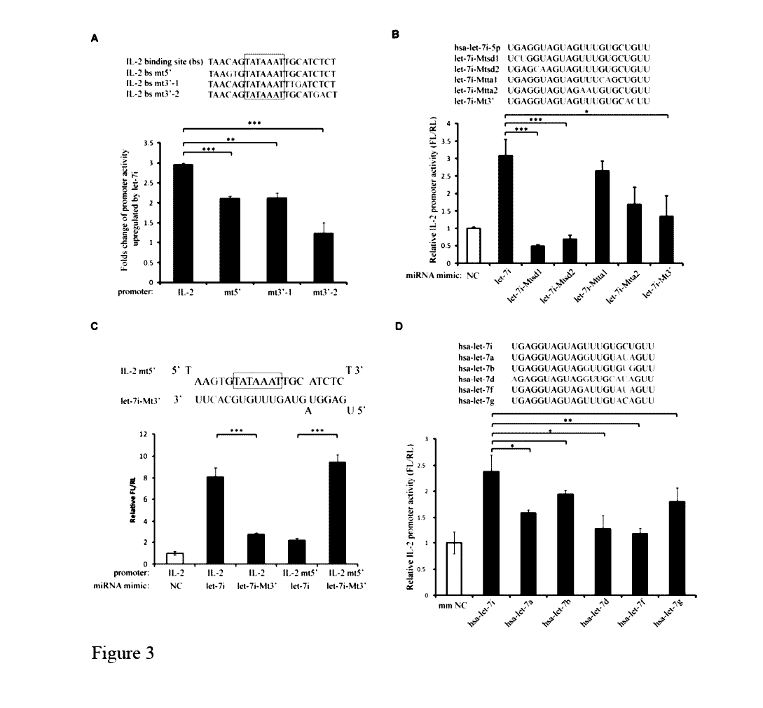

Enhancing the expression levels of IL-2 by miRNA hsa-let-7i is sequence-specific, and its regulatory effect is in positive relation to the complementarity between miRNA and TATA box sequences of gene promoter. When mutations introduced into combining sites on IL-2 promoter, effects of upregulating transcriptional activity of IL-2 promoter by hsa-let-7i are affected (FIG. 3A). Accordingly, upon mutating hsa-let-7i sequence, effects of upregulating transcriptional activity of IL-2 promoter by hsa-let-7i are also affected (FIG. 3B). When simultaneously mutating combining sites on IL-2 promoter and hsa-let-7i sequence, and rematching them both, effects of upregulating transcriptional activity of IL-2 promoter by hsa-let-7i are restored (FIG. 3C). Other members of Let-7i family including hsa-let-7a, hsa-let-7b, hsa-let-7d, hsa-let-7f, and hsa-let-7g are all able to up-regulate the transcriptional activity of IL-2 promoter, whose effects are related to its similarity with hsa-let-7i in sequence (FIG. 3 D). These results demonstrate that upregulating the transcriptional activity of IL-2 promoterby hsa-let-7i is sequence-specific.

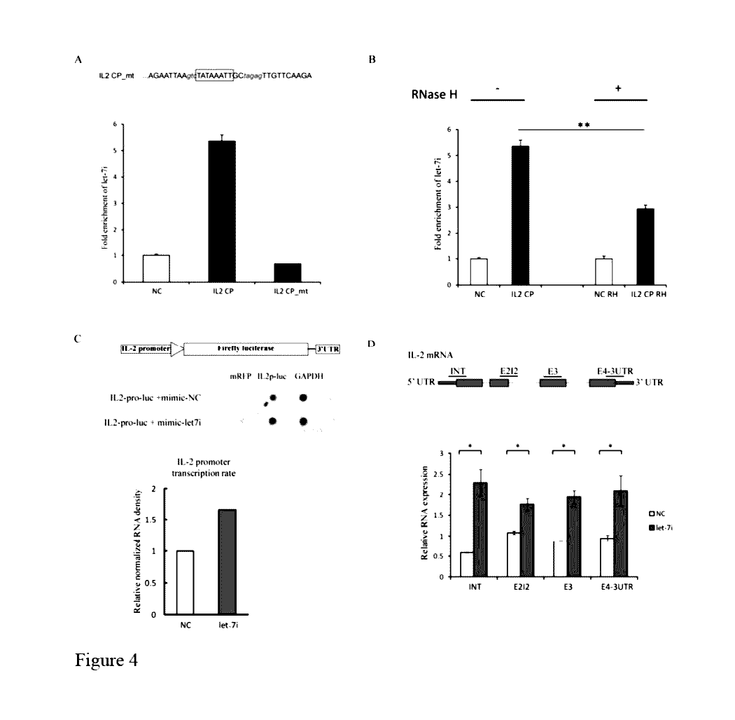

MiRNAs can directly combine with TATA box sequence of gene promoter, and result in increased initiation rate of gene transcription. Combining experiments in vitro show that hsa-let-7i can combine with the wildtype IL-2 promoter DNA, rather than a mutated IL-2 promoter DNA (FIG. 4A). After treatment with RNase H, hsa-let-7i enriched by IL-2 promoter DNA reduces, suggesting that those two are combined through DNA:RNA hybrid chain (FIG. 4B). Analysis of initiation rate of gene transcription shows that hsa-let-7i enhances transcription initiation rate of IL-2 gene promoter (FIG. 4C), and the transcription elongation rate increases with it (FIG. 4D).

### Example 2

MiRNA hsa-miR-138, hsa-miR-92a, hsa-let-7c and hsa-miR-181d sequence-specifically up-regulates the transcriptional activities of genes of insulin, calcitonin, histone (H4A1) and c-myc by targeting the TATA box, respectively.

2.1 Preparation Method

Computational Prediction of miRNAs and Targeting Sites Thereof

MiRNAs and combining sites on gene promoters are predicted and selected using the method according to Example 1.

Plasmids, and microRNA Mimics

Reporter vectors of the promoters such as human insulin, CALCA, c-myc, H4-A1 and so on are constructed according to the method of Example 1. The precursor sequence of hsa-mir-181d is inserted into PLKO.1 vector (Sigma). Wildtype hsa-miR-138, hsa-miR-92a, hsa-let-7c and the corresponding negative control are purchased from Ribobio (Guangzhou, China).

Analysis of Dual-Luciferase Reporter

According to the method of Example 1, the luciferase reporter vector of said gene promoter and luciferase vector of Renilla are transfected, and precursor vector of miRNA or control vector, or miRNA mimics or small interfering RNAs and the corresponding negative controls are cotransfected. The activities of dual-luciferase are examined after 24-48 hours.

2.2 Structure

The structure of the resultant miRNA is as follows:

hsa-miR-138:
5′ agcugguguugugaaucaggccg 3′
hsa-miR-92a:
5′ uauugcacuugucccggccugu 3′
hsa-let-7c:
5′ ugagguaguagguuguaugguu 3′
hsa-miR-181d-5p:
5′ aacauucauuguugucggugggu 3′

2.3 Confirmation of Effects and Application

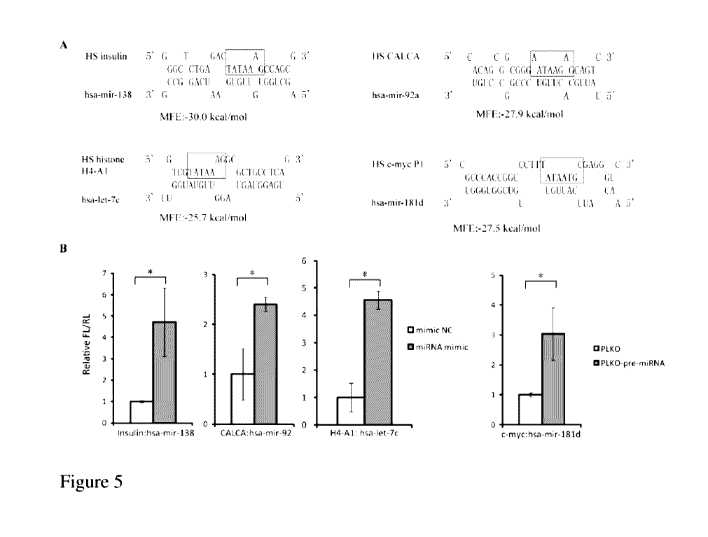

Computational predictions indicate that miRNAs including hsa-miR-138, hsa-miR-92a, hsa-let-7c and hsa-miR-181d are capable of combining with the TATA box sequences of insulin, calcitonin, histone (H4A1) and c-myc genes respectively (FIG. 5A). The results of dual-luciferase reporter show that hsa-miR-138, hsa-miR-92a, hsa-let-7c and hsa-miR-181d are capable of enhancing the transcriptional activities of genes of insulin, calcitonin, histone (H4A1) and c-myc respectively (FIG. 5B).

### Example 3

### Artificially-Synthesized Small Interfering RNAs Targeting TATA Box of Gene Specifically Enhancing the Transcriptional Activity of 78.9% of Genes

3.1 Preparation Methods

Design of Sequences of Small Interfering RNAs Targeting TATA Box

Within a range of 20 bases extending both upstream and downstream from TATA box sequence serving as a center, three small interfering RNAs complementary to TATA box in sequence are designed.

Plasmids, and Small Interfering RNA Mimics

The reporter vectors of promoters of human IL-2, insulin, LHB, POMC, NPPA, IL6, HIV-1 virus, H4A1, APOE, CIRBP, BCL2L12, RHO, CALCA, GAPD and HBB are constructed according to the method of Example 1. Small interfering RNAs and the corresponding negative control are synthesized by Ribobio (Guangzhou, China).

Analysis of Dual-Luciferase Reporter

According to the method of example 1, the luciferase reporter vector of gene promoter and luciferase vector of Renilla are transfected, and precursor vector of miRNA or control vector, or miRNA mimics or small interfering RNAs and the corresponding negative controls are cotransfected. The activities of dual-luciferase are examined after 24-48 hours.

Structure

The structure of the resultant miRNA is as follows:

IL-2TATAcen:
5′ agaugcaauuuauacuguu 3′
INS-TATAcen:
5′ cgcuggcuuuauagucuca 3′
LHB-TATAcen:
5′ uaucuggcuauauaccucg 3′
POMC-TATAcen:
5′ ucuguccuuauauacuugc 3′
NPPA-TATAcen:
5′ cgccucuuuuuauagcccc 3′
IL6-TATAcen:
5′ uggaaaccuuauuaagauu 3′
HIV-TATAcen:
5′ cagcugcuuauauguagca 3′
H4A1-TATAup:
5′ Ucuuuauacgacaguuggc 3′
APOE-TATAcen:
5′ uuguccaauuauagggcuc 3′
CIRBP-TATAup:
5′ ucuuauauacgcuuccgcc 3′
BCL2L12-TATAcen:
5′ uacaaacuuuauuaguucg 3′
RHO-TATAcen:
5′ uccccagacccuuauaaag 3′
CALCA-TATAup:
5′ ugcucuuauucccgccgcu 3′
GAPD-TATAdn:
5′ ugcucaauuuauagaaacc 3′
HBB-TATAcen:
5′ ugcccugacuuuuaugccc 3′

3.3 Confirmation of Effects and Application

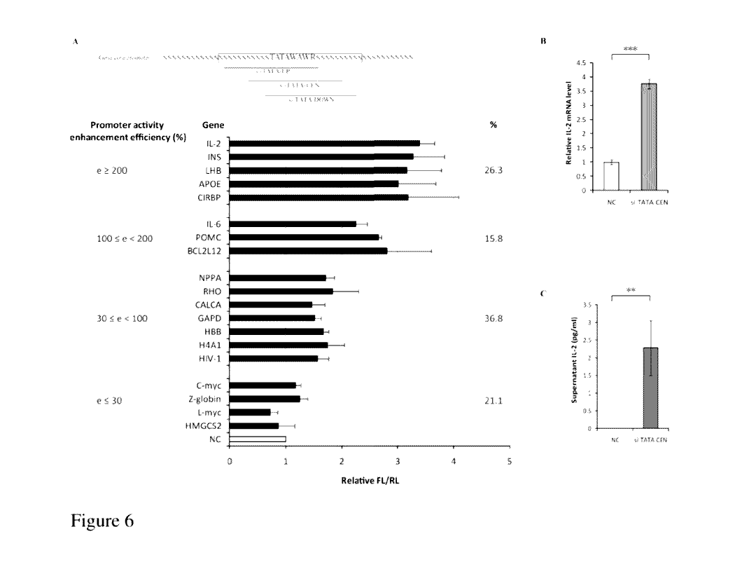

According to the preparation method, we design 1-3 small interfering RNAs with respect to 19 genes containing TATA box (FIG. 6A). The results of dual-luciferase reporter show that the transcriptional activities of 5 genes are enhanced by two folds or more by means of the siRNAs; the transcriptional activities of 3 genes are enhanced by one fold by means of the siRNAs; and the transcriptional activities of 7 genes are enhanced by more than 30% by means of the siRNAs. The transcription activates of a total of 15 genes are increased by more than 30%, accounting for 78.9% of total verified genes (FIG. 6A). The verifications of the expression level of mRNAs and proteins show that the siRNAs can significantly enhance the expression level of interleukin-2 mRNA (FIG. 6B) and protein expression (FIG. 6C).

### Example 4

### Artificially-Synthesized Small Interfering RNAs Targeting TATA Box of Gene Specifically Enhancing the Transcriptional Activity of the Non-TATA Box Genes

4.1 Preparation Methods

Design of siRNA Sequence Targeting the Core Promoter of Non-TATA Box Genes

In the range of 1-50 bp upstream from the transcription starting site (TSS) of a gene, a siRNA targeting the core promoter is designed.

Plasmids, and siRNA Mimics

The reporter vectors of human NGF and APOBR promoters are constructed according to the method of Example 1. Small interfering RNAs and the corresponding negative control are synthesized by Ribobio (Guangzhou, China).

Analysis of Dual-Luciferase Reporter

According to the method of example 1, luciferase reporter vector of gene promoter and luciferase vector of Renilla are transfected, and precursor vector of miRNA or control vector, or miRNA mimics or small interfering RNAs and the corresponding negative controls are cotransfected. The activities of dual-luciferase are examined after 24-48 hours.

Transfection of Small Interfering RNAs and Real Time Fluorescent Quantitative PCR Examination

According to the method of example 1, the small interfering RNAs and the corresponding negative control are transfected into HEK293T cell lines, and mRNA expression levels of genes of AKT1, CDC25A, ERK2, BMI-1 and JNK are examined using real time fluorescent quantitative PCR after 48 hours.

4.2 Structure

The structure of the resultant miRNA is as follows:

si-NGF:
5′ gagcugcucucacacaggcuu 3′
si-APOBR:
5′ uaaugaccguccccacccacc 3′
si-AKT1:
5′ uccgccccgcgcccgcccc 3′
si-CDC25A:
5′ ugcccagcuccggguagca 3′
si-ERK2:
5′ uccggcgggcgggcggagg 3′
si-BMI-1:
5′ ugaggcgggcgggcggggg 3′
si-JNK:
5′ ugucaccgcgcacgcccgc 3′

4.3 Confirmation of Effects and Application

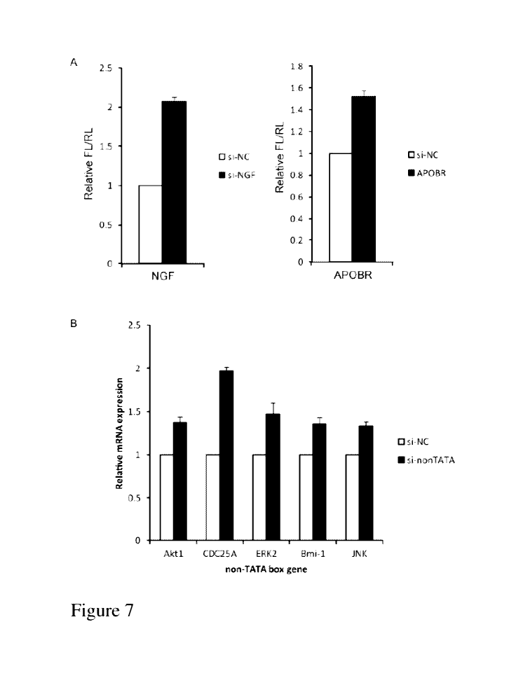

According to the preparation method, we design a small interfering RNA with respect to each of core promoters of seven non-TATA box genes. The results of dual-luciferase reporter show that the siRNAs enhance the transcriptional activities of genes of NGF, and APOBR (FIG. 7A). The verifications of the expression levels of mRNAs show that siRNAs could significantly enhance the mRNA expression levels of AKT1, CDC25A, ERK2, BMI-1 and JNK (FIG. 7B).

What is claimed:

1. A preparation method of small RNAs for upregulating expression of endogenous gene, including the following steps:
S1. identification of target sites of small RNAs,
which are in the range of 20 bases extending upstream and downstream respectively from a TATA box sequence serving as a center, when the TATA box sequence is contained in a promoter, and
which are sequences of 1-50 bases upstream from a starting site for the gene transcription, when the TATA box sequence isn't contained in the promoter;
S2. design and synthesis of small RNAs,
antisense sequence of the small RNAs being complementary to sequence of the target site, to obtain a core sequence; and
S3. modifying the resultant core sequence of S2, so that the small RNAs contain the following structures:
double-stranded siRNA having a length of 19 bases and a dTdT pendant at 3′ terminal, double-stranded siRNA having a length less than or more than 19 bases, microRNA synthesized in vitro, single-stranded or double-stranded antisense RNA synthesized in vitro, or chemically modified siRNA, microRNA and antisense RNA synthesized in vitro.

2. Application of a small RNA obtained from the method according to claim 1 in regulating transcriptional activities of genes and medicines thereof.

3. The application according to claim 2, characterized in that, said regulating transcriptional activity of gene means combining with the TATA box sequence or other core promoters to thereby enhance the expression of gene.

4. A small RNA prepared according to the preparation method of claim 1.

5. The small RNA according to claim 4, characterized in that, said small RNA is hsa-let-7i, hsa-miR-138, hsa-miR-92a, hsa-let-7c, hsa-miR-181d, IL-2TATAcen, INS-TATAcen, LHB-TATAcen, POMC-TATAcen, NPPA-TATAcen, IL6-TATAcen, HIV-TATAcen, H4A1-TATAup, APOE-TATAcen, CIRBP-TATAup, BCL2L12-TATAcen, RHO-TATAcen, CALCA-TATAup, GAPD-TATAdn, or HBB-TATAcen,
a gene sequence of said hsa-let-7i is shown as SEQ NO: 1,
a gene sequence of said hsa-miR-138 is shown as SEQ NO: 2,
a gene sequence of said hsa-miR-92a is shown as SEQ NO: 3,
a gene sequence of said hsa-let-7c is shown as SEQ NO: 4,
a gene sequence of said hsa-miR-181d is shown as SEQ NO: 5,
a gene sequence of said IL-2TATAcen is shown as SEQ NO: 6,
a gene sequence of said INS-TATAcen is shown as SEQ NO: 7,
a gene sequence of said LHB-TATAcen is shown as SEQ NO: 8,
a gene sequence of said POMC-TATAcen is shown as SEQ NO: 9,
a gene sequence of said NPPA-TATAcen is shown as SEQ NO: 10,
a gene sequence of said IL6-TATAcen is shown as SEQ NO: 11,
a gene sequence of said HIV-TATAcen is shown as SEQ NO: 12,
a gene sequence of said H4A1-TATAup is shown as SEQ NO: 13,
a gene sequence of said APOE-TATAcen is shown as SEQ NO: 14,
a gene sequence of said CIRBP-TATAup is shown as SEQ NO: 15,
a gene sequence of said BCL2L12-TATAcen is shown as SEQ NO: 16,
a gene sequence of said RHO-TATAcen is shown as SEQ NO: 17,
a gene sequence of said CALCA-TATAup is shown as SEQ NO: 18,
a gene sequence of said GAPD-TATAdn is shown as SEQ NO: 19, and
a gene sequence of said HBB-TATAcen is shown as SEQ NO: 20.

6. Application of the small RNA according to claim 5 in regulating transcriptional activities of genes and medicines thereof.

7. The application according to claim 6, characterized in that, said regulating transcriptional activity of gene means enhancing the transcriptional activities of genes of IL-2, insulin, calcitonin, histone, c-myc, LHB, POMC, NPPA, IL6, HIV-1 virus, H4A1, APOE, CIRBP, BCL2L12, RHO, CALCA, GAPD, or HBB.


SEQUENCE LISTING (27 sequences)

SEQ ID NO: 1 - RNA, 22 nt, Artificial Sequence
  miRNA
ugagguaguaguuugugcuguu

SEQ ID NO: 2 - RNA, 23 nt, Artificial Sequence
  miRNA
agcugguguugugaaucaggccg

SEQ ID NO: 3 - RNA, 22 nt, Artificial Sequence
  miRNA
uauugcacuugucccggccugu

SEQ ID NO: 4 - RNA, 22 nt, Artificial Sequence
  miRNA
ugagguaguagguuguaugguu

SEQ ID NO: 5 - RNA, 23 nt, Artificial Sequence
  miRNA
aacauucauuguugucggugggu

SEQ ID NO: 6 - RNA, 19 nt, Artificial Sequence
  siRNA
agaugcaauuuauacuguu

SEQ ID NO: 7 - RNA, 19 nt, Artificial Sequence
  siRNA
cgcuggcuuuauagucuca

SEQ ID NO: 8 - RNA, 19 nt, Artificial Sequence
  siRNA
uaucuggcuauauaccucg

SEQ ID NO: 9 - RNA, 19 nt, Artificial Sequence
  siRNA
ucuguccuuauauacuugc

SEQ ID NO: 10 - RNA, 19 nt, Artificial Sequence
  siRNA
cgccucuuuuuauagcccc

SEQ ID NO: 11 - RNA, 19 nt, Artificial Sequence
  siRNA
uggaaaccuuauuaagauu

SEQ ID NO: 12 - RNA, 19 nt, Artificial Sequence
  siRNA
cagcugcuuauauguagca

SEQ ID NO: 13 - RNA, 19 nt, Artificial Sequence
  siRNA
ucuuuauacgacaguuggc

SEQ ID NO: 14 - RNA, 19 nt, Artificial Sequence
  siRNA
uuguccaauuauagggcuc

SEQ ID NO: 15 - RNA, 19 nt, Artificial Sequence
  siRNA
ucuuauauacgcuuccgcc

SEQ ID NO: 16 - RNA, 19 nt, Artificial Sequence
  siRNA
uacaaacuuuauuaguucg

SEQ ID NO: 17 - RNA, 19 nt, Artificial Sequence
  siRNA
uccccagacccuuauaaag

SEQ ID NO: 18 - RNA, 19 nt, Artificial Sequence
  siRNA
ugcucuuauucccgccgcu

SEQ ID NO: 19 - RNA, 19 nt, Artificial Sequence
  siRNA
ugcucaauuuauagaaacc

SEQ ID NO: 20 - RNA, 19 nt, Artificial Sequence
  siRNA
ugcccugacuuuuaugccc

SEQ ID NO: 21 - RNA, 21 nt, Artificial Sequence
  miRNA
gagcugcucucacacaggcuu

SEQ ID NO: 22 - RNA, 21 nt, Artificial Sequence
  miRNA
uaaugaccguccccacccacc

SEQ ID NO: 23 - RNA, 19 nt, Artificial Sequence
  miRNA
uccgccccgcgcccgcccc

SEQ ID NO: 24 - RNA, 19 nt, Artificial Sequence
  miRNA
ugcccagcuccggguagca

SEQ ID NO: 25 - RNA, 19 nt, Artificial Sequence
  miRNA
uccggcgggcgggcggagg

SEQ ID NO: 26 - RNA, 19 nt, Artificial Sequence
  miRNA
ugaggcgggcgggcggggg

SEQ ID NO: 27 - RNA, 19 nt, Artificial Sequence
  miRNA
ugucaccgcgcacgcccgc